(19) United States
(12) Patent Application Publication
(10) Pub. No.: US 2007/0129330 A1
(43) Pub. Date: Jun. 7, 2007

(54) 1,3-Diaza-dibenzo[e,h]azulenes for the treatment of central nervous system diseases and disorders

(75) Inventors: Mladen Mercep, Zagreb (HR); Milan Mesic, Zagreb (HR); Renata Rupcic, Zagreb (HR); Dijana Pesic, Sibenik (HR)

Correspondence Address: DARBY & DARBY P.C., P. O. BOX 5257, NEW YORK, NY, 10150-5257, US

(73) Assignee: Pliva-Istrazivacki Institut d.o.o., Zagreb, Croatia (HR)

(21) Appl. No.: 10/595,936

(22) PCT Filed: Nov. 19, 2004
(86) PCT No.: PCT/HR04/00056

§ 371 Date: Aug. 11, 2006

(30) Foreign Application Priority Data
Nov. 21, 2003 (HR) P20030958A

(51) Int. Cl. A61K 31/695; A61K 31/55; A61K 31/4188

(52) U.S. Cl. 514/63; 514/215; 514/393

(57) ABSTRACT

The present invention relates to the use of compounds from the group of 1,3-diaza-dibenzo[e,h]azulenes and of their pharmaceutically acceptable salts and solvates for the for the use in the treatment and prevention of diseases, damages and disorders of the central nervous system (CNS) caused by disorders of the neurochemical equilibrium of biogenic amines or other neurotransmitters.

This application is a National Stage under 35 U.S.C. §371 of PCT International Application No. PCT/HR2004/000056, filed Nov. 19, 2004, which claims the benefit under 35 U.S.C. §19(e) of prior Croatian Application No. P20030958 A, filed Nov. 21, 2003. The International Application was published in English on Jun. 2, 2005 as WO 2005/049016 A1 under PCT Article 21(2).

### FIELD OF THE INVENTION

The present invention relates to the use of compounds from the class of 1,3-diaza-dibenzo[e,h]azulenes as well as of their pharmacologically acceptable salts and solvates for the use in the treatment and prevention of diseases, damages and disorders of the central nervous system (CNS) caused by disorders of the neurochemical equilibrium of biogenic amines or other neurotransmitters.

### BACKGROUND OF THE INVENTION

Irregularities in the steady state of biogenic amines (serotonin, norepinephrine, dopamine) and of other neurotransmitters and their receptors that are part of central neurotransmitter system in the CNSmay be the cause of various mental diseases, damages and disorders (e.g. depression, schizophrenia, manic behavior and similar). Pathological changes in the CNS caused by disorders of neurotransmitter concentration may occur due to an unbalanced (too big or too small) synthesis, irregularities in storing, releasing, metabolizing and/or reabsorption of biogenic amines and/or certain neurotransmitters.

The results of investigations directed to the understanding of pathogenesis of mental disorders have shown that a disorder in the serotonin equilibrium plays an important role in various diseases. The monoamine-deficiency hypothesis was one of the first explanations, wherein the symptoms of depression were connected to a reduction in the neurotransmission of monoamines, especially serotonin (5-HT) and noradrenaline, which was also confirmed by neurochemical tests as well as by a successful treatment of the patients with substances increasing monoaminergic neurotransmission (Expert Opin. Investig. Drugs 2003, 12, 531-543). In addition to the serotonergic and noradrenergic systems, a very important role in the CNS function disorders is also played by the dopaminergic system. The understanding of the exact role and of the interactions of these neurotransmitter systems is made rather difficult by the great number of receptor subtypes and their pharmacological complexity. Thus, it has been observed that e.g. dopaminergic neurotransmission is regulated by 5-HT_{2A} receptors (L. G. Spampinato, J. Neurochem. 2000, 74, 693-701) and hence 5-HT₂A receptors may also be the target receptors in treating diseases and disorders, in whose pathology an important role is played by a disorder of the function of the dopaminergic system (psychoses and various addictions).

Glutamate receptors play a vital role in the mediation of excitatory synaptic transmission as one of the major excitatory neurotransmitters in the central nervous system (CNS). It is widely accepted that σ1 receptor ligands can modulate neurotransmission mediated by the central neurotransmitter systems, including glutamatergic/NMDA (F.P. Monnet, G. Debonnel, J.-L. Junien, C. de Montigny, Eur. J. Phannacol.,1990, 179, 441-445). Many pharmacological and physiological actions have been attributed to the ⊕1 receptor. These include the regulation of IP3 receptors and calcium signaling at the endoplasmic reticulum, mobilization of cytoskeletal adaptor proteins, modulation of nerve growth factor-induced neurite sprouting, modulation of neurotransmitter release and neuronal firing, modulation of potassium channels as a regulatory subunit, alteration of psychostimulant-induced gene expression, and blockade of spreading depression. Behaviorally, the σ1 receptor is involved in learning and memory, psychostimulant-induced sensitization, cocaine-induced conditioned place preference, schizophrenia and pain perception. Thus, it is hypothesized that the σ1 receptor, at least in part, is intracellular amplifier creating a supersensitized state for signal transduction in the biological system.

For treatment of pathological CNS disorders and particularly for mental disorders, the most frequently applied medicines are polycyclic compounds (benzodiazepines, tricyclic and tetracyclic antidepressants, monoamino oxidase (MAO) inhibitors, selective inhibitors of serotonin reabsorption etc.).

A new area in pharmacotherapy was opened by introducing the novel tetracyclic antidepressant mianserin (Claghorn, J.; Lesem, M. D. Prog. Drug Res. 1996, 46, 243-262; Sperling, W.; Demling, J. Drugs Today 1997, 33, 95-102). Numerous tetracyclic derivatives showing pharmacological action in the treatment of the disorders of the neurochemical equilibrium in the CNS are disclosed in the literature. WO 99/19317, WO 97/38991 and U.S. Pat. No. 6,511,976 describe the manufacture of tetracyclic derivatives containing tetrahydrofuran ring and the use thereof as substances having antipsychotic, cardiovascular and gastrokinetic actions. U.S. Pat. No. 4,145,434 discloses the manufacture of dibenzo(cyclohepta-, oxepino-, thiepino-)pyrrolidine and dibenzopyrrolidinoazepine derivatives as well as the use thereof as substances having a potential CNS action. The manufacture and an antidepressive action of some 1,2-diaza-dibenzoazepines are disclosed in EP 0063525. The manufacture and a potential anxiolytic action of some tetracyclic isooxazolidine derivatives are disclosed as well (Drugs Fut. 2002, 27, Suppl. A: C41; Drugs Fut. 2002, 27, Suppl. A: P182, WO 96/14320, WO 96/14321). The introduction of a piperidine ring into a tetracyclic structure containing an oxepine ring resulted in the formation of the molecule Org-4428 showing an antidepressive action (Sperling, W.; Demling, J. Drugs Today 1997, 33, 95-102). The molecule Org-5222 containing a pyrrolidine ring fused to an oxepine nucleus is described as a potential anxiolytic and antipsychotic (Sperling, W.; Demling, J. Drugs Today 1997, 33, 95-102).

Further there are described derivatives of 1,3-diaza-dibenzo[e,h]azulenes with different substituents in 2-position, e.g. trifluoromethyl, pyridyl, naphthyl, phenyl and substituted phenyl and salts thereof as a novel class of compounds with antiinflammatory action (U.S. Pat. Nos. 3,711,489, 3,781,294 and CA 967,573). As substances with antiinflammatory action there are described also 1,3-diaza-dibenzo[e,h]azulenes with alkylthio substituents in 2-position (U.S. Pat. No. 4,198,421, EP 372445, WO 91/18885).

However, the known medicines for pathological CNS disorders and particularly for mental disorders are associated with a wide range of adverse effects. Thus, there is a need for a safe and effective treatment of diseases and disorders of CNS.

In our earlier International publication WO 03/099823, herein incorporated by reference in its entirety as amended with a letter dated Jun. 13, 2004, we disclose compounds of 1,3-diaza-dibenzo[e,h]azulene class, of their pharmaceutically acceptable salts and solvates, process and intermediates for preparation thereof as well as their antiinflammatory effects especially to the inhibition of tumor necrosis factor-α (TNF-α) production and the inhibition of interleukin-1 (IL-1) production along with their analgesic action.

We have now surprisingly found that compounds from the class of 1,3-diaza-dibenzo[e,h]azulenes as described in aforementioned specification are effective in the treatment of diseases and disorders of CNS. The present compounds differ structurally from the art-known tetracyclic compounds acting upon CNS by, an unsaturated tetracyclic structure since they contain an imidazole ring as the fourth ring, whereas the art-known tetracyclic compounds acting upon CNS (WO 99/19317, WO 97/38991; Sperling, W.; Demling, J. Drugs Today 1997, 33, 95-102) contain at least one saturated ring in their structure, and are further distinguished by valuable pharmacological and physicochemical properties.

According to our knowledge, the use of 1,3-diaza-dibenzo[e,h]azulenes and of their pharmaceutically acceptable salts and solvates disclosed in our earlier International publication WO 03/099823 for the use in the treatment and prevention of diseases, damages and disorders of the central nervous system caused by disorders of neurochemical steady state has hitherto been neither disclosed nor suggested.

### SUMMARY OF THE INVENTION

The present invention provides for the effective treatment and prevention of diseases, damages and disorders of the central nervous system caused by disorders of equilibrium of biogenic amines. Accordingly, the invention relates to the use of compounds from the class of 1,3-diaza-dibenzo[e,h]azulenes of the general formula I 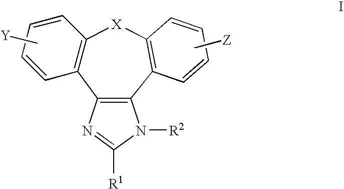
wherein
X is CH₂ or a heteroatom selected from a group consisting of O, S, S(═O), S(═O)₂ and NR^{a}, wherein R^{a} is hydrogen or a substituent selected from the group consisting of C₁-C₃-alkyl, (preferably methyl or ethyl), C₁-C₃-alkanoyl (preferably acetyl), C₁-C₇-alkoxycarbonyl, (preferably methoxycarbonyl or tert. -butoxycarbonyl), C₇-C₁₀-arylmethoxycarbonyl (preferably benzyloxycarbonyl), C₇-C₁₀-aroyl (preferably benzoyl), C₇-C₁₀-arylalkyl (preferably benzyl), C₃-C₇-alkylsilyl (preferably trimethylsilyl) and C₅-C₁₀-alkylsilylalkoxyalkyl (preferably trimethylsilylethoxymethyl);
Y and Z independently from each other mean one or more identical or different substituents linked to any available carbon atom selected from the group consisting of hydrogen, halogen, C₁-C₄-alkyl, C₂-C₄-alkenyl, C₂-C₄-alkynyl, halo-Cl-C₄-alkyl, hydroxy, Cl-C₄-alkoxy, trifluoromethoxy, C₁-C₄-alkanoyl, amino, amino-C₁-C₄-alkyl, N-(C₁-C₄-alkyl)amino, N,N-di(C₁-C₄-alkyl)amino, thiol, C₁-C₄-alkylthio, sulfonyl, Cl-C₄-alkylsulfonyl, sulfinyl, C₁-C₄-alkylsulfinyl, carboxy, C₁-C₄-alkoxycarbonyl, cyano and nitro;
R¹ is CHO, CH═CHOCOCH₃, (CH₂)ₘOH wherein m is an integer from 1 to 3, or a substituent of the formula II: 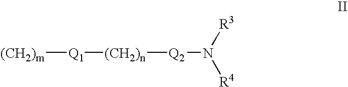
   wherein
   R³ and R⁴ simultaneously or independently from each other are hydrogen, C₁-C₄-alkyl, aryl having the meaning of an aromatic ring as well as fused aromatic rings containing one ring with at least 6 carbon atoms or two rings with a total of 10 carbon atoms and with alternating double bonds between carbon atoms; or together with N are heterocycle or heteroaryl wherein heterocycle relates to five-member or six-member fully saturated or partly unsaturated heterocycle group containing at least one hetero atom selected from the group consisting of O, S and N and where said heterocycle can be optionally substituted with one or two substituents which are selected from halogen, C₁-C₄ alkyl, cyano, nitro, hydroxy, C₁-C₄ alkoxy, thiol, C₁-C₄ alkylthio, amino, N-(C₁-C₄) alkylamino, N,N-di(C₁-C₄-alkyl)-amino, sulfonyl, C₁-C₄ alkylsulfonyl, sulfinyl, C₁-C₄ alkylsulfinyl; and wherein heteroaryl relates to aromatic and partially aromatic groups of a monocyclic or bicyclic ring with 4 to 12 carbon atoms and at least one of them being heteroatom selected from the group consisting of O, S and N and where said heteroaryl can be optionally substituted with one or two substituents which are selected from halogen, C₁-C₄ alkyl, cyano, nitro, hydroxy, C₁-C₄ alkoxy, thiol, C₁-C₄ alkylthio, amino, N-(C₁-C₄) alkylamino, N,N-di(C₁-C₄-alkyl)-amino, sulfonyl, C₁-C₄ alkylsulfonyl, sulfinyl, C₁-C₄ alkylsulfinyl;
   m is an integer from 1 to 3;
   n is an integer from 0 to 3;
   Q₁ and Q₂ independently from each other are oxygen, sulfur or a group: 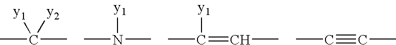
      wherein substituents
   y₁ and y₂ independently from each other are hydrogen, halogen, C₁-C₄-alkyl optionally substituted with one, two, three or more substituents selected from the group consisting of halogen atom, hydroxy, C₁-C₄ alkoxy, thiol, C₁-C₄ alkylthio, amino, N-(C₁-C₄) alkylamino, N,N-di(C₁-C₄-alkyl)-amino, sulfonyl, C₁-C₄ alkylsulfonyl, sulfinyl and C₁-C₄ alkylsulfinyl; or monocyclic or bicyclic aryl group having from 6 to 10 carbon atoms and altering double bond and said group can be optionally substituted with one or two substituents selected from the group consisting of fluoro, chloro, C₁-C₄ alkyl, cyano, nitro, hydroxy, C₁-C₄ alkoxy, thiol, C₁-C₄ alkylthio, amino, N-(C₁-C₄) alkylamino, N,N-di(C₁-C₄-alkyl)-amino, sulfonyl, C₁-C₄ alkylsulfonyl, sulfinyl, C₁-C₄ alkylsulfinyl and can be linked to the rest of the molecule by any available carbon atom via direct bond or via C₁-C₄ alkylene group; hydroxy; C₁-C₄-alkoxy; C₁-C₄-alkanoyl; thiol; C₁-C₄-alkylthio; sulfonyl; C₁-C₄-alkylsulfonyl; sulfinyl; C₁-C₄-alkylsulfinyl; cyano; nitro; or together form a carbonyl or imino group;
R₂ is hydrogen, an optionally substituted C₁-C₇-alkyl or aryl wherein an optionally substituted alkyl or aryl are as defined above, C₁-C₇-alkanoyl, C₁-C₇-alkoxycarbonyl, C₇-C_{10-arylalkyloxycarbonyl, C}₇-C₁₀-aroyl, C₇-C₁₀-arylalkyl, C₃-C₇-alkylsilyl, C₆H₅CH₂CH₂ and CH₂OCH₂CH₂Si(CH₃)₃;
   and of their pharmaceutically acceptable salts and solvates is pharmaceutical formulations for the treatment and prevention of diseases, damages and disorders of the central nervous system caused by disorders of neurochemical equilibrium of biogenic amines or other neurotransmitters.

### DETAILED DESCRIPTION OF THE INVENTION

The term “halo”, “hal” or “halogen” relates to a halogen atom which may be fluorine, chlorine, bromine or iodine (most preferably chlorine or bromine).

The term “alkyl” relates to alkyl groups with the meaning of alkanes-wherefrom radicals are derived, which radicals may be straight, branched or cyclic or a combination of straight and cyclic ones and branched and cyclic ones. The preferred straight or branched alkyls are e.g. methyl, ethyl, propyl, isopropyl, butyl, sec-butyl and tert-butyl. The preferred cyclic alkyls are e.g. cyclopentyl or cyclohexyl.

The term “haloalkyl” relates to alkyl groups which must be substituted with at least one halogen atom. The most frequent haloalkyls are e.g. chloromethyl, dichloromethyl, trifluoromethyl or 1,2-dichloropropyl.

The term “alkenyl” relates to alkenyl groups having the meaning of hydrocarbon radicals, which may be straight, branched or cyclic or are a combination of straight and cyclic ones or branched and cyclic ones, but having at least one carbon-carbon double bond. The most frequent alkenyls are ethenyl, propenyl, butenyl or cyclohexenyl.

The term “alkynyl” relates to groups having the meaning of ydrocarbon radicals, which are straight or branched and contain at least one and at most two carbon-carbon triple bonds. The most frequent alkynyls are e.g. ethynyl, propynyl or butynyl.

The term “alkoxy” relates to straight or branched chains of alkoxy group. Examples of such groups are methoxy, propoxy, prop-2-oxy, butoxy, but-2-oxy or methylprop-2-oxy.

The term “aryl” relates to groups having the meaning of an aromatic ring, e.g. phenyl, as well as to fused aromatic rings. Aryl contains one ring with at least 6 carbon atoms or two rings with a total of 10 carbon atoms and with alternating double (resonant) bonds between carbon atoms. The most frequently used aryls are e.g. phenyl or naphthyl. In general, aryl groups may be linked to the rest of the molecule by any available carbon atom via a direct bond or via a C₁-C₄ alkylene group such as methylene or ethylene.

The term “heteroaryl” relates to groups having the meaning of aromatic and partially aromatic groups of a monocyclic or bicyclic ring with 4 to 12 carbon atoms, at least one of them being a hetero atom such as O, S or N, and the available nitrogen atom or carbon atom is the binding site of the group to the rest of the molecule either via a direct bond or via a C₁-C₄ alkylene group defined earlier. Examples of this type are thiophenyl, pyrrolyl, imidazolyl, pyridinyl, oxazolyl, thiazolyl, pyrazolyl, tetrazolyl, pirimidinyl, pyrazinyl, quinolinyl or triazinyl.

The term “heterocycle” relates to five-member or six-member, fully saturated or partly unsaturated heterocyclic groups containing at least one hetero atom such as O, S or N, and the available nitrogen atom or carbon atom is the binding site of the group to the rest of the molecule either via a direct bond or via a C₁-C₄ alkylene group defined earlier. The most frequent examples are morpholinyl, piperidinyl, piperazinyl, pyrrolidinyl, pirazinyl or imidazolyl.

The term “alkanoyl” group relates to straight chains of acyl group such as formyl, acetyl or propanoyl.

The term “aroyl” group relates to aromatic acyl groups such as benzoyl.

The term “optionally substituted alkyl” relates to alkyl groups which may be optionally additionally substituted with one, two, three or more substituents. Such substituents may be halogen atom (preferably fluorine or chlorine), hydroxy, C₁-C₄ alkoxy (preferably methoxy or ethoxy), thiol, C₁-C₄ alkylthio (preferably methylthio or ethylthio), amino, N-(C₁-C₄) alkylamino (preferably N-methylamino or N-ethylamino), N,N-di(C₁-C₄-alkyl)-amino (preferably dimethylamino or diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

The term “optionally substituted alkenyl” relates to alkenyl groups optionally additionally substituted with one, two or three halogen atoms. Such substituents may be e.g. 2-chloroethenyl, 1,2-dichloroethenyl or 2-bromo-propene-1-yl.

The term “optionally substituted aryl, heteroaryl or heterocycle” relates to aryl, heteroaryl or heterocyclic groups which may be optionally additionally substituted with one or two substituents. The substituents may be halogen (preferably chlorine or fluorine), C₁-C₄ alkyl (preferably methyl, ethyl or isopropyl), cyano, nitro, hydroxy, C₁-C₄ alkoxy (preferably methoxy or ethoxy), thiol, C₁-C₄ alkylthio (preferably methylthio or ethylthio), amino, N-(C₁-C₄) alkylamino (preferably N-methylamino or N-ethyl amino), N,N-di(C₁-C₄-alkyl)-amino (preferably N,N-dimethylamino or N,N-diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

When X is NR^{a}, R^{a} is hydrogen or group selected from the C₁-C₃-alkyl (preferably methyl or ethyl), C₁-C₃-alkanoyl (preferably formyl or acetyl), C₁-C₇-alkoxycarbonyl (preferably methoxycarbonyl or tert-butoxycarbonyl), C₇-C₁₀-arylalkyloxycarbonyl (preferably benzyloxycarbonyl), C₇-C₁₀-aroyl (preferably benzoyl), C₇-C₁₀-arylalkyl (preferably benzyl), C₃-C₇-alkylsilyl (preferably trimethylsilyl) or C₅-C₁₀-alkylsilylalkoxyalkyl (preferably trimethylsilylethoxymethyl).

When R³ and R⁴ together with N are heteroaryl or heterocycle, this means that such heteroaryl or heterocycle has at least one carbon atom replaced by a nitrogen atom through which the groups are linked to the rest of the molecule. Examples of such groups are morpholine-4-yl, piperidine-1-yl, pyrrolidine-1-yl, imidazole-1-yl or piperazine-1-yl.

Depending upon the nature of particular substituents, the compounds of the formula I may have geometric isomers and one or more chiral centres so that there can exist enantiomers or diastereoisomers. The present invention also relates to use of such isomers and mixtures thereof, including racemates.

The present invention also relates to all possible tautomeric forms of particular compounds of the formula I.

Whenever used hereinafter, the term “compounds of formula I” or “compounds of the present invention” is meant to also include the pharmaceutically acceptable addition salts and solvates.

In one embodiment of the present invention preferred compounds of formula I are those wherein X is O, S, or NR^{a}, wherein R^{a} is hydrogen or substituent selected from the group consisting of C₁-C₃-alkyl (preferably methyl, ethyl, propyl or isopropyl), C₁-C₃-alkanoyl (preferably formyl or acetyl), C₇-C₁₀-aroyl (preferably benzoyl) and C₇-C₁₀-arylalkyl (preferably benzyl).

In another embodiment of the present invention preferred compounds of formula I are those wherein Y and Z independently from each other mean one or more identical or different substituents linked to any available carbon atom selected from the group consisting of hydrogen, fluorine, chlorine, bromine, C₁-C₄-alkyl (preferably methyl, ethyl, propyl or isopropyl), halo-C₁-C₄-alkyl (preferably trifluoromethyl), hydroxy, C₁-C₄-alkoxy (preferably methoxy), trifluoromethoxy, C₁-C₄-alkanoyl (preferably formyl or acetyl), amino, amino-C₁-C₄-alkyl (preferably aminomethyl), N-(C₁-C₄-alkyl)amino (preferably N-methyl or N-ethyl), N,N-di(C1-C₄-alkyl)amino (preferably dimethylamino or diethylamino), thiol, Cl-C₄-alkylthio (preferably methylthio), cyano and nitro.

In yet another embodiment of the present invention preferred compounds of formula I are those wherein R¹ is CHO, CH═CHOCOCH₃, (CH₂)ₘOH wherein m is an integer from 1 to 3;
or a substituent represented with the formula II: 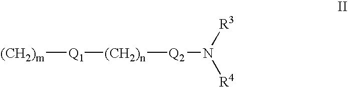
wherein
R³ and R⁴ simultaneously or independently from each other represent hydrogen, C₁-C₄-alkyl, aryl wherein aryl is as defined above; or together with N are heterocycle or heteroaryl selected from the group consisting of morpholine-4-yl, piperidine-1-yl, pyrrolidine-1-yl, imidazole-1-yl and piperazine-1-yl;
m is an integer from 1 to 3;
n is an integer from 0 to 3;
Q₁ and Q₂ independently from each other are oxygen or CH₂ group.

In still another embodiment of the present invention preferred compounds of formula I are those wherein R² is hydrogen, an optionally substituted C₁-C₄-alkyl wherein an optionally substituted alkyl are as defined above, C₁-C₇-alkanoyl, C₇-C₁₀-aroyl, C₇-C₁₀-arylalkyl, C₆H₅CH₂CH₂ and CH₂OCH₂CH₂Si(CH₃)₃.

In yet another embodiment of the present invention the specifically preferred compounds of formula I are:
1-methyl-1H-8-oxa-1,3-diaza-dibenzo [e,h]azulene-2-carbaldehyde;
1-methyl-1H-8-thia-1,3-diaza-dibenzo [e,h]azulene-2-carbaldehyde;
1-phenethyl-1H-8-oxa-1,3-diaza-dibenzo[e,h]azulene-2-carbaldehyde;
1-phenethyl-1H-8-thia-1,3-diaza-dibenzo [e,h]azulene-2-carbaldehyde;
1-(2-trimethylsilyl-ethoxymethyl)-1H-8-oxa-1,3-diaza-dibenzo[e,h]azulene-2-carbaldehyde;
1-(2-trimethylsilyl-ethoxymethyl)-1H-8-thia-1,3-diaza-dibenzo[e,h]azulene-2-carbaldehyde;
5-chloro-1-(2-trimethylsilyl-ethoxymethyl)-1H-8-oxa-1,3-diaza-dibenzo[e,h]azulene-2-carbaldehyde;
11-chloro-1-(2-trimethylsilyl-ethoxymethyl)-1H-8-oxa-1,3-diaza-dibenzo [e,h]azulene-2-carbaldehyde;
5-chloro-1-(2-trimethylsilyl-ethoxymethyl)-1H-8-thia-1,3-diaza-dibenzo[e,h]azulene-2-carbaldehyde;
11-chloro-1-(2-trimethylsilyl-ethoxymethyl)-1H-8-thia-1 ,3-diaza-dibenzo [e,h]azulene-2-carbaldehyde;
3-(1-phenethyl-1H-8-thia-1,3-diaza-dibenzo[e,h]azulen-2-yl)-acrylic acid methyl ester;
(1-methyl-1H-8-oxa-1,3-diaza-dibenzo[e,h]azulen-2-yl)-methanol;
(1-methyl-1H-8-thia-1,3-diaza-dibenzo [e,h]azulen-2-yl)-methanol;
(1-phenethyl-1H-8-oxa-1,3-diaza-dibenzo [e,h]azulen-2-yl)-methanol;
(1-phenethyl-1H-8-thia-1,3-diaza-dibenzo [e,h]azulen-2-yl)-methanol;
[1-(2-trimethylsilyl-ethoxymethyl)-1H-8-oxa-1,3-diaza-dibenzo[e,h]azulen-2-yl]-methanol;
[1-(2-trimethylsilyl-ethoxymethyl)-1H-8-thia-1,3-diaza-dibenzo[e,h]azulen-2-yl]-methanol;
[5-chloro-1-(2-trimethylsilyl-ethoxymethyl)-1H-8-oxa-1,3-diaza-dibenzo[e,h]azulen-2-yl]-methanol
[11-chloro-1-(2-trimethylsilyl-ethoxymethyl)-1H-8-oxa-1,3-diaza-dibenzo[e,h]azulen-2-yl]-methanol;
[5-chloro-1-(2-trimethylsilyl-ethoxymethyl)-1H-8-thia-1,3-diaza-dibenzo[e,h]azulen-2-yl]-methanol;
[1-chloro-1-(2-trimethylsilyl-ethoxymethyl)-1H-8-thia-1,3-diaza-dibenzo[e,h]azulen-2-yl]-methanol;
3-(1-phenethyl-1H-8-thia-1,3-diaza-dibenzo[e,h]azulen-2-yl)-propane-1-ol;
dimethyl-[2-(1-methyl-1H-8-oxa-1,3-diaza-dibenzo[e,h]azulen-2-ylmethoxy)-ethyl]-amine;
dimethyl-[3-(1-methyl-1H-8-oxa-1,3-diaza-dibenzo[e,h]azulen-2-ylmethoxy)-propyl]-amine;
dimethyl-[2-(1-methyl-1H-8-thia-1,3-diaza-dibenzo[e,h]azulen-2-ylmethoxy)-ethyl]-amine;
dimethyl-[3-(1-methyl-1H-8-thia-1,3-diaza-dibenzo[e,h]azulen-2-ylmethoxy)-propyl]-amine;
dimethyl-[2-(1-phenethyl-1H-8-oxa-1,3-diaza-dibenzo[e,h]azulen-2-ylmethoxy)-ethyl]-amine;
dimethyl-[3-(1-phenethyl-1H-8-oxa-1,3-diaza-dibenzo[e,h]azulen-2-ylmethoxy)-propyl]-amine;
dimethyl-[2-(1-phenethyl-1H-8-thia-1,3-diaza-dibenzo[e,h]azulen-2-ylmethoxy)-ethyl]-amine;
dimethyl-[3-(1-phenethyl-1H-8-thia-1,3-diaza-dibenzo[e,h]azulen-2-ylmethoxy)-propyl]-amine;
dimethyl-{2-[1-(2-trimethylsilyl-ethoxymethyl)-1H-8-oxa-1, 3-diaza-dibenzo[e,h]azulen-2-ylmethoxy]-ethyl}-amine;
dimethyl-[2-(1H-8-oxa-1,3-diaza-dibenzo[e,h]azulen-2-ylmethoxy)-ethyl]-amine;
dimethyl-{3-[1-(2-trimethylsilyl-ethoxymethyl)-1H-8-oxa-1,3-diaza-dibenzo [e,h]azulen-ylmethoxy]-propyl}-amine;
dimethyl-[3-(1H-8-oxa-1,3-diaza-dibenzo[e,h]azulen-2-ylmethoxy)-propyl]-amine;
3-[1-(2-trimethylsilyl-ethoxymethyl)-1H-8-oxa-1,3-diaza-dibenzo[e,h]azulen-2-ylmethoxy]-propylamine;
3-(1H-8-oxa-1,3-diaza-dibenzo[e,h]azulen-2-ylmethoxy)-propylamine;
dimethyl-{2-[1-(2-trimethylsilyl-ethoxymethyl)-1H-8-thia-1,3-diaza-dibenzo[e,h]azulen-2-ylmethoxy]-ethyl}-amine;
dimethyl-[2-(1H-8-thia-1,3-diaza-dibenzo[e,h]azulen-2-ylmethoxy)-ethyl]-amine;
dimethyl-{3-[1-(2-trimethylsilyl-ethoxymethyl)-1H-8-thia-1,3-diaza-dibenzo[e,h]azulen-2-ylmethoxy]-propyl}-amine;
dimethyl-[3-(1H-8-thia-1,3-diaza-dibenzo[e,h]azulen-2-ylmethoxy)-propyl]-amine;
{3-[5-chloro-1-(2-trimethylsilyl-ethoxymethyl)-1H-8-oxa-1,3-diaza-dibenzo[e,h]azulen-2-ylmethoxy]-propyl}-dimethyl-amine;
[3-(5-chloro-1H-8-oxa-1,3-diaza-dibenzo[e,h]azulen-2-ylmethoxy)-propyl]-dimethyl-amine;
3-[5-chloro-1-(2-trimethylsilyl-ethoxymethyl)-1H-8-oxa-1,3-diaza-dibenzo[e,h]azulen-2-ylmethoxyl-propylamine;
3-(5-chloro-1H-8-oxa-1,3-diaza-dibenzo[e,h]azulen-2-ylmethoxy)-propylamine;
{2-[11-chloro-1-(2-trimethylsilyl-ethoxymethyl)-1H-8-oxa-1,3-diaza-dibenzo[e,h]azulen-2-ylmethoxy]-ethyl}-dimethyl-amine;
[2-(11-chloro-1H-8-oxa-1,3-diaza-dibenzo[e,h]azulen-2-ylmethoxy)-ethyl]-dimethyl-amine;
{3-[11-chloro-1-(2-trimethylsilyl-ethoxymethyl)-]H-8-oxa-1,3-diaza-dibenzo[e,h]azulen-2-ylmethoxy]-propyl}-dimethyl-amine;
[3-(11-chloro-1H-8-oxa-1,3-diaza-dibenzo[e,h]azulen-2-ylmethoxy)-propyl]-dimethyl-amine;
{2-[5-chloro-1-(2-trimethylsilyl-ethoxymethyl)-1H-8-thia-1,3-diaza-dibenzo [e,h]azulen-2-ylmethoxy]-ethyl}-dimethyl-amine;
[2- (5-chloro-1H-8-thia-1,3-diaza-dibenzo[e,h]azulen-2-ylmethoxy)-ethyl]-dimethyl-amine;
{3-[5-chloro-1-(2-trimethylsilyl-ethoxymethyl)-1H-8-thia-1,3-diaza-dibenzo[e,h]azulen-2-ylmethoxyl]-propyl}-dimethyl-amine;
[3-(5-chloro-1H-8-thia-1,3-diaza-dibenzo[e,h]azulen-2-ylmethoxy)-propyl]-dimethyl-amine; and dimethyl-{3-[3-(1-phenethyl-1H-8-thia-1,3-diaza-dibenzo[e,h]azulen-2-yl)-propoxy]-propyl}-amine.

Generally, the compounds of 1,3-diaza-dibenzo[e,h]azulene class, their pharmaceutically acceptable salts and solvates represented by the formula I can be prepared by the processes set forth in our earlier International publication WO 03/099823, herein incorporated by reference in its entirety as amended in the letter of Jun. 13, 2004.

The compounds of the present invention are especially effective in treating those diseases and disorders where the neurochemical equilibrium of biogenic amines such as serotonin, norepinephrine and dopamine was disturbed and which may be caused by unbalanced (too big or too small) synthesis, irregularities in storing, releasing, metabolizing and/or reabsorption of a certain neurotransmitter.

It has been found that the compounds of the present invention exhibit a significant binding affinity and have a high degree of selectivity to serotonin receptors, especially to 5-HT_{2A} and 5-HT_{2C}, as well as for the σ1 receptor.

In one embodiment of the present invention the compound of formula I, or salt, or solvate thereof show binding affinity to 5-HT_{2A} and 5-HT_{2C} serotonin receptors in the concentration expressed as an IC₅₀ value less than 1 μM and having Kᵢ value less than 1 μM.

In another embodiment of the present invention the compound of formula I, or salt, or solvate thereof show binding affinity to 5-HT_{2A} serotonin receptor in the concentration expressed as an IC₅₀ value less than about 200 nM and having Kᵢ value less than about 100 nM.

In yet another embodiment of the present invention the compound of formula I, or salt, or solvate thereof show binding affinity to 5-HT_{2C} serotonin receptor in the concentration expressed as an IC₅₀ value less than about 200 nM and having Kᵢ value less than about 100 nM.

It has been found that the compounds of the present invention exhibit a significant binding affinity to the σ1 receptor.

In one embodiment of the present invention the compound of formula I, or salt, or solvate thereof show binding affinity to the σ1 receptor in the concentration expressed as an IC₅₀ value less than 1 μM and having Kᵢ value less than 1 μM.

In another embodiment of the present invention the compound of formula I, or salt, or solvate thereof show binding affinity to the σ1 receptor in the concentration expressed as an IC₅₀ value less than about 200 nM and having Kᵢ value less than about 100 nM.

Since serotonin receptors are crucial in pathophysiology of a series of CNS disorders (directly or indirectly by participating in the activation of some other neurotransmitter e.g. dopamine and/or receptor), the compounds of the present invention may be used is pharmaceutical formulations for the treatment and prevention of diseases, damages and disorders, wherein biogenic amines and their receptors play an important role.

In view of the above explained favourable biological properties of the compounds of the present invention administration of the therapeutically effective amount of a compound of formula I provides an effective method of treatment of CNS diseases and disorders associated with fewer side effects due to their improved selectivity towards the σ1 receptor and the 5-HT₂A and 5-HT_{2C} serotonin receptors.

Pharmaceutical Compositions

In general, the compounds of the present invention may be used is pharmaceutical formulations that are used as antidepressants, anxiolytics, antipsychotics or as drugs for treating migraine.

Further, the compounds of the present invention may be used is pharmaceutical formulations for the treatment and prevention of diseases and disorders which are the result of disorders of neurochemical equilibrium in the central nervous system such as e.g. depression and modest depression, anxiety, bipolar disorders, sleeping disorders, sexual disorders, psychoses, borderline psychoses, schizophrenia, migraine, personality disorders and obsessive-compulsive disorders, social phobias or panic attacks, organic mental disorders in children, aggression, memory disorders and personality disorders in elderly people, addiction, obesity, bulimia and similar disorders, snoring, premenstrual troubles.

Likewise, these compounds may be used in the treatment and/or prevention of CNS damage caused by trauma, brain stroke, neurodegenerative diseases, cardiovascular disorders such as high blood pressure, thrombosis, infarct and similar diseases as well as in gastrointestinal disorders.

The effective dose of the active substance of the present invention and of a pharmaceutically acceptable salt or solvate thereof depends on the efficacy of the compound of the general formula I, on the nature and the severity of the disease and the disorder of CNS as well as on the body weight of the patient treated and may be from 0.001-10 mg/kg body weight. In any case a unit dose for an adult of an average weight of 70 kg is understood to be 0.07-1000 mg of the compound of the general formula I or of a pharmaceutically acceptable salt or solvate thereof. A unit dose may be administered once or several times daily, e.g. 2, 3 or 4 times daily, most frequently 1 to 3 times daily.

The present invention more specifically relates to an effective dose of the compounds which bind to serotonin, sigma, adrenergic, dopamine or muscarinic receptors and/or act as inhibitors of reabsorption of one or more biogenic amines (serotonin, dopamine, norepinephrine).

The term “salts” can include acid addition salts or addition salts of free bases. Examples of acids which may be employed to form pharmaceutically acceptable acid addition salts include but are not limited to salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al. “Pharmaceutical Salts,” J. of Pharma. Sci., 1977; 66:1).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Preferred pharmaceutically acceptable salts according to invention relate to salts of hydrobromic, hydrochloric, perchloric, sulfuric, maleic, fumaric, tartaric, citronic, benzoic, mandelic, methanesulfonic, benzenesulfonic, oxalic, p-toluenesulfonic, 2-naphthalenesulfonic and phosphoric acid.

Pharmaceutically acceptable solvates formed by the compounds represented by formula I or their salts relate to hydrates, ethanolates and similar (most frequently hydrates).

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopoeia or other generally recognized pharmacopeias for use in mammals, and more particularly in humans.

Further, the present invention relates to a pharmaceutical formulation containing an effective non-toxic dose of the compounds of the present invention as well as pharmaceutically acceptable carriers or solvents.

The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. However, since memantine is highly soluble, aqueous solutions are preferred. Suitable pharmaceutical carriers are described in “Remington's Pharmaceutical Sciences” by E. W. Martin, 18th Edition. Particularly preferred for the present invention are carriers suitable for immediate-release, i.e., release of most or all of the active ingredient over a short period of time, such as 60 minutes or less, and make rapid absorption of the drug possible.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The pharmaceutical formulations are obtained by blending a therapeutically active amount of a certain substance as the active ingredient with a pharmaceutically acceptable carrier, which may have different forms depending on the desired administration route. These pharmaceutical formulations especially relate to oral, sublingual, rectal, percutaneous or parenteral administration route.

Pharmaceutical formulations may be manufactured using conventional pharmaceutical auxiliaries and manufacture routes. Forms for oral administration may be syrups, capsules, tablets and similar forms where usual solid carriers are inert substances such as lactose, starch, glucose, methylcellulose, magnesium stearate, dicalcium phosphate, mannitol and similar, and usual liquid oral auxiliaries include ethanol, glycerol, water and similar. All auxiliaries may be optionally blended with disintegrants, diluents, granulating agents, wetting agents, binders and similar by using conventional methods. Parenteral forms may be manufactured by using water or some other sterile carrier. When for the manufacture of oral formulations some of the common liquid carriers e.g. water, glycol, oils, alcohols and similar are used, the formulation may be in the form of syrup, emulsion, soft gelatine capsules or sterile injectable liquids e.g. ampoules, or of non-aqueous liquid suspensions. When for the manufacture of oral formulations a solid carrier such as starch, sugar, kaolin, wetting agents, binders, disintegrants and similar is used, the formulation may be in the form of a powder, capsule, tablet, hard gelatine capsules or granules that may be administered in capsules, and the amount of the solid carrier may vary (most frequently from 1 mg to 1 g). Due to their easy use, tablets and capsules are the most convenient oral formulations wherein a solid carrier is used. For parenteral formulations the carrier is mostly sterile water, though other ingredients may be contained therein as well in order to improve solubility. For the manufacture of injectable solutions, sodium chloride solution, glucose solution or a mixture thereof is used. Injectable solutions may also contain a component for a delayed release of the active component. Convenient oils that may be used for this purpose are e.g. arachic oil, sesame oil, cottonseed oil, corn oil, soybean oil, synthetic glycerol esters of long-chain fatty acids or a mixture of some of said oils. Injectable suspensions may be manufactured in such a way that a suitable liquid carrier used is blended with a suspending agent. In formulations convenient for percutaneous administration, as a carrier there is understood a substance improving the penetration of the active substance and/or a suitable wetting agent, which may be combined with a suitable additive of any provenience, which additives do not cause harmful effects on skin. Said additives may facilitate the skin administration and/or may be used in the manufacture of the desired formulations, which may be applied in various ways e.g. transdermally, spot-on, or in the form of an ointment.

To improve the solubility and/or stability of the present compounds, in pharmacological formulations there may be used α-, β- or γ-cyclodextrins or derivatives thereof, especially hydroxyalkyl substituted cyclodextrins i.e. 2-hydroxypropyl-β-cyclodextrin. Cosolvents such as e.g. alcohols may also improve the solubility and/or stability of the present compounds in various pharmaceutical formulations.

“Treating” or “treatment” of a state, disorder or condition includes:
(1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition,
(2) inhibiting the state, disorder or condition, i.e., arresting or reducing the development of the disease or at least one clinical or subclinical symptom thereof, or
(3) relieving the disease, i.e., causing regression of the state, disorder or condition or at least one of its clinical or subclinical symptoms.

The benefit to a subject to be treated is either statistically significant or at least perceptible to the patient or to the physician.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated. Dosages and administration regimen can be adjusted depending on the age, sex, physical condition as well as the benefit achieved by applying the compounds of the present invention and the side effects in the patient or the mammalian subject to be treated and the judgement of the physician, as is appreciated by those skilled in the art.

The term host or subject in need thereof as used herein refers to a mammal preferably a human.

Biological Assays

The effect of the compounds of the present invention on the neurochemical steady state was determined by in vitro investigations such as a radionuclide-marked radioligand binding assay for 5-HT_{2A} (Bonhaus D. W. Br. J. Pharmacol. 1995, 115:622; Saucier C. J. Neurochem. 1997, 68:1998) and 5-HT_{2C} receptors (Wolf W.A. J. Neurochem. 1997, 69:1449), in vitro binding assay for the σ1 receptor (Thomson W. and Donn R. Arthritis Res. 2002, 4: 302-306) and by in vivo investigations in a tail suspension test (Vogel H. G. and Vogel W. H. Drug Discovery and Evaluation Pharmacological Assays, Springer 1997, 304), in amphetamine-induced hyperlocomotion in mice (Millan M. J. et al, 1998 J. Pharmacol. Exp. Ther. 287: 167-186), in a forced swim test in mice (Porsolt R. D. et al. Arch. Int. Pharmacodyn. 1977, 229:327-336), in meta-chlorophenyl piperazine (m-CPP) test on rats (Drug Dev. Res. 1989, 18:119-144), and in apomorphine, tryptamine and norepinephrine (ATN) test in rats (Arch. Int. Pharmacodyn. 1977, 227:238-253).

In Vitro Method for Determining Affinity for Binding to 5-HT_{2A} and 5-HT_{2C} Receptors

A small concentration of a radioligand having a great affinity for binding to a receptor was incubated with a tissue sample enriched with a certain receptor (1-5 mg of tissue) in a buffered medium (0.2-5 mL). Recombinant human HT_{2A} and HT_{2C} receptors were expressed in CHO-K1 or COS-7 cells and were also used for competitive binding. During incubation the radioligand bound to the receptor. When a binding balance was achieved, the receptors to which the radioligand was bound were separated from those to which said ligand was not bound, and the radioactivity of the receptor/radioligand complex was measured. The interaction of the tested compounds with receptors was tested in competitive binding experiments. Various concentrations of tested compounds were added to the incubation mixture containing a prepared tissue enriched with corresponding receptors and the radioligand. The radioligand binding was inhibited by the test compounds proportionally to the affinity of a certain compound for the receptor and to the concentration of the compound.

The radioligand used for the determination of binding to 5-HT_{2A} receptor was [³H]-ketanserin and the tissue used was human cortex or recombinant 5-HT_{2A} receptor expressed in CHO-K1 cells. The radioligand used for the determination of binding to 5-HT_{2C} receptor was [³H]-mesulergine and the tissue used was choroid plexus or recombinant 5-HT_{2C} receptor expressed in CHO-K1 cells.

Compounds showing IC₅₀ and Kᵢ values lower than 1 μM, were considered to be active. Compounds: dimethyl-{3-[2-(1H-8-thia-1,3-diaza-dibenzo[e,h]azulen-2-yl)-phenoxy]-propyl}-amine, [3-(11-chloro-1H-8-thia-1,3-diaza-dibenzo[e,h]azulen-2-ylmethoxy)-propyl]-dimethyl-amine,11chloro-1H-8-oxa-1,3-diaza-dibenzo[e,h]azulene and dimethyl-[2-(1-methyl-1H-8-thia-1,3-diaza-dibenzo[e,h]azulen-2-ylmethoxy)-ethyl]-amine showed binding affinity to 5-HT_{2A} and 5-HT_{2C} serotonin receptors expressed as IC₅₀ value less than 200 nM and Ki value less than 100 nM. It is anticipated that similar results will be observed for other compounds of the invention.

In Vitro Method for Determining Binding Affinity to the σ1 Receptor

Jurkat cell were grown in medium, RPMI supplemented with 10% fetal bovine serum, 100U/ml penicillin and 100 μg/ml streptomycin, collected and their suspension homogenized. After centrifugation, membrane fraction was separated, resuspended in phosphate buffer (pH=7.5) and stored in small aliquots in liquid nitrogen until use. Binding of different radiolabeled ligands to Jurkat cell membranes was measured as described previously (Ramamoorthy et al., 1995). To characterize the 6 binding sites in the Jurkat cell line, [³H]haloperidol as first used as the ligand. Haloperidol is a high affinity ligand to both type 1 and type 2 σ-receptors. The binding assays were done using Jurkat cell membranes in the presence of [³H]haloperidol (10 nM) alone to determine the total binding, and in the presence of [³H]haloperidol (10 nM) and unlabeled haloperidol (10 μM) to determine the nonspecific binding. Membranes were incubated with ligands in phosphate buffer for 3 hours at room temperature. After filter had been washed, radioactivity associated with the filter was determined by liquid scintillation spectrometry.

Compounds showing IC₅₀ and Kᵢ values lower than 1 μM, were considered to be active. It is anticipated that similar results will be observed for other compounds of the invention.

Forced Swim Test in Mice

Male CD1 mice of the weight of 20-25 g were used for the experiment. Groups of 10 animals were treated with the test compounds, imipramine (positive control) or the vehicle (negative control) by per os by gavage 30 min prior to testing to determine efficacy. On the day of the experiment the animals were placed into a glass cylinder (height 18.2 cm, diameter 13.3 cm) filled with water warmed to 22° C. to the height of 10 cm. The immobility defined as the end of the struggling of the animal and the beginning of floating, wherein the movements were reduced to those indispensable for the animal to keep its head over the water surface, started to be recorded after two minutes and then it was monitored during 4 minutes. The percentage of animals showing a passive behaviour was calculated and compared with a control group treated with a carrier.

The compounds that in a dose of 10 mg/kg reduced the immobility of animals for 30% and more over the control group were considered to be active. It is anticipated that similar results will be observed for other compounds of the invention.

Tail Suspension Test in Mice

Male Balb/cJ mice of the weight of 20-25 g were used for the experiment. Groups of 9 animals were treated with the test compounds, imipramine (positive control) or the vehicle (negative control) by intraperitoneal injection, subcutaneous injection or per oral by gavage 30 min prior to testing to measure potential antidepressant activity. Mice were suspended from their tails at a height of about 90 cm and were observed for 5 minutes. The mice hanging fully motionless for 1 minute during the observation period were defined as depressive. In animals treated with a substance having an antidepressive action the period of immobility was shortened.

The percentage of animals showing a passive behaviour was calculated and compared with a control group treated with a vehicle. Significance of results was analysed using Fischer's exact test.

The compounds that in a dose of 10 mg/kg reduced the immobility of animals for 40% and more over a control group were considered to be active. It is anticipated that similar results will be observed for other compounds of the invention.

Amphetamine-Induced Hyperlocomotion in Mice

Male Swiss OFA mice of a weight 30-35 g were treated with either vehicle (saline) or test compounds 30 minutes prior to hyperlocomotion induction. Dexamphetamine sulphate was administered intraperitoneally at 2 mg/kg. Thirty minutes later, animals were placed in a wooden box 80×80 cm in a room with low light intensity (100 lux) for locomotor activity recording. Locomotor activity was determined during a 30 min period using a video image analyzer. Total duration of movement, occurence of movement and total distance travelled were measured. Haloperidol was tested at the dose of 0,25 mg/kg (prepared in 0,5% methylcellulose) and served as reference substance.

Compounds were considered as active if in a dose of 10 mg/kg reduced amphetamine-induced hyperlocomotion in experimental animals for 30% and more when compared to vehicle treated control group. It is anticipated that similar results will be observed for other compounds of the invention.

Meta-Chlorophenyl Piperazine (m-CPP) Test on Rats

The tested substance was administered to rats per os 1 hour before the test and m-CPP in a dose of 1 mg/kg was administered intravenously 15 minutes before the test. At the beginning of the experiment the treated animals were subjected to an open field test on rats (Drug Dev. Res. 1989, 18, 119-144): the apparatus consisted of an open box having the dimensions 80×65×35 cm, which in one wall had an opening with a diameter of 10 cm, by which it was connected to a non-illuminated compartment having the dimensions 25×21×21 cm, and the opening was illuminated by a light source (IR source or Kleverluxo®; 12V/20 W) from the distance of 66 cm; one hour after administering the tested substance, the animals were placed in the dark (non-illuminated) compartment so that their heads were turned away from the illuminated exit and the passing of the animals from the dark compartment to the bright one was measured for 10 minutes.

As an active dose of the substance there was defined a dose at which the effect induced by m-CPP was reduced for 40% and more. It is anticipated that similar results will be observed for other compounds of the invention.

Apomorphine, Tryptamine, Norepinephrine (ATN) Test in Rats

At the beginning of the experiment (t=0) the animals were injected intravenously by 1.25 mg/kg of apomorphine, then by 40 mg/kg of tryptamine (t=60 minutes) and by 1.25 mg/kg of norepinephrine (t=90 minutes). There were watched a state of exceptional agitation and normal behaviour during 60 minutes in apomorphine test, then bilateral (two-sided) clonic convulsions of back paws (legs) and a general tremor of the body in tryptamine test (observation period 5 minutes) and lethality during 120 minutes after the injection in norepinephrine test.

The percentage of animals showing a passive behaviour was calculated and compared with a control group treated with a carrier.

The compounds which in a dose of 10 mg/kg reduced the period of duration of observed effects (mobility) for 40% over a control group were considered to be active in in vivo testings. It is anticipated that similar results will be observed for other compounds of the invention.

Some of the present compounds tested in the above assays showed an action in at least two of said tests, though these results represent only an illustration of the biological action of the compounds and do not limit the present invention in any way.

1. A method of treating a disease, damage or disorder of the central nervous system associated with a disorder of neurochemical equilibrium of a biogenic amine or other neurotransmitter, comprising administering to a subject in need thereof a compound of formula I 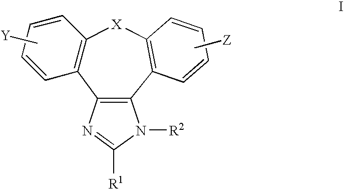
wherein
X is selected from the group consisting of CH₂,O, S, S(═O), S(═O)₂ and NR^{a}, wherein R^{a} is selected from the group consisting of hydrogen, C₁-C₃-alkyl, Cₗ-C₃-alkanoyl, C₁-C₇-alkoxycarbonyl, C₇-C₁₀-arylmethoxycarbonyl, C₇-C₁₀-aroyl, C₇-C₁₀-arylalkyl, C₃-C₇-alkylsilyl and C₅-C₁₀-alkylsilylalkoxyalkyl;
Y and Z are each independently selected from the group consisting of hydrogen, halogen, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N-(C₁-C₄-alkyl)amino, N,N-di(C₁-C₄-alkyl)amino, thiol, C₁-C₄-alkylthio, sulfonyl, C₁-C₄-alkylsulfonyl, sulfinyl, C₁-C₄-alkylsulfinyl, carboxy, C₁-C₄-alkoxycarbonyl, cyano and nitro;
R¹ is selected from the group consisting of CHO, CH═CHOCOCH₃, (CH₂)ₘOH wherein m represents is an integer from 1 to 3, and a substituent of the formula II: 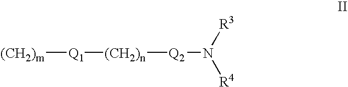
wherein
R³ and R⁴ are each independently, C₁-C₄-alkyl or aryl; or
R³ and R⁴ taken together with the nitrogen atom to which they are attached form a heterocycle or heteroaryl group that is optionally substituted with one or two substituents which are selected from the group consisting of halogen, C₁-C₄ alkyl, cyano, nitro, hydroxy, C₁-C₄ alkoxy, thiol, C₁-C₄ alkylthio, amino, N-(C₁-C₄) alkylamino, N,N-di(C₁-C₄-alkyl)-amino, sulfonyl, C₁-C₄ alkylsulfonyl, sulfinyl, and C₁-C₄ alkylsulfinyl;
m is an integer from 1 to 3;
n is an integer from 0 to 3;
Q₁ and Q₂ are each independently selected from the group consisting of oxygen, sulfur, 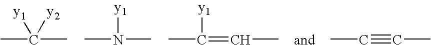
wherein substituents
y₁ and y₂ are each independently selected from the group consisting of hydrogen, halogen, C₁-C₄-alkyl optionally substituted with one, or more substituents selected from the group consisting of halogen, hydroxy, C₁-C₄ alkoxy, thiol, C₁-C₄ alkylthio, amino, N-(C₁-C₄) alkylamino, N,N-di(C₁-C₄-alkyl)-amino, sulfonyl, C₁-C₄ alkylsulfonyl, sulfinyl and C₁-C₄ alkylsulfinyl; hydroxy, C₁-C₄-alkoxy, C₁-C₄-alkanoyl, thiol, C₁-C₄-alkylthio, sulfonyl, C₁-C₄-alkylsulfonyl, sulfinyl, C₁-C₄-alkylsulfinyl, cyano, nitro, and an aryl group wherein said ary group is optionally substituted with one or two substituents selected from the group consisting of fluoro, chloro, C₁-C₄ alkyl, cyano, nitro, hydroxy, C₁-C₄ alkoxy, thiol, C₁-C₄ alkylthio, amino, N-(C₁-C₄) alkylamino, N,N-di(C₁-C₄-alkyl)-amino, sulfonyl, C₁-C₄ alkylsulfonyl, sulfinyl, C₁-C₄ alkylsulfinyl and is linked to the rest of the molecule via a direct bond or a C₁-C₄ alkylene group; or
y₁ and y₂ taken together with the carbon atom to which they are attached form a carbonyl group or an imino group;
R² is selected from the group consisting of hydrogen, a C₁-C₇-alkyl group optionally substituted with one or more substituents selected from the group consisting of halogen, hydroxy, C₁-C₄ alkoxy, thiol, C₁-C₄ alkylthio, amino, N-(C₁-C₄) alkylamino, N,N-di(C₁-₄-alkyl)-amino, sulfonyl, C₁-C₄ alkylsulfonyl, sulfinyl and C₁-C₄ alkylsulfinyl; an aryl group optionally substituted with one or two substituents selected from the group consisting of fluoro, chloro, C₁-C₄ alkyl, cyano, nitro, hydroxy, C₁-C₄alkoxy, thiol, C₁-C₄ alkylthio, amino, N-(C₁-C₄) alkylamino, N,N-di(C₁-C₄-alkyl)-amino, sulfonyl, C₁-C₄ alkylsulfonyl, sulfinyl, C₁-C₄ alkylsulfinyl; C₁-C₇-alkanoyl, C₁-C₇-alkoxycarbonyl, C₇-C₁₀-arylalkyloxycarbonyl, C₇-C₁₀-aroyl, C₇-C₁₀-arylalkyl, C₃-C₇-alkylsilyl, C₆H₅CH₂CH₂ and CH₂OCH₂CH₂Si(CH₃)₃;
and of their a pharmaceutically acceptable salt or solvate thereof.

2. The method of claim 1, wherein the biogenic amine is serotonin, norepinephrine or dopamine.

3. The method of claim 1, wherein the neurotransmitter is glutamate.

4. The method of claim 1, wherein the compound of formula I regulates the synthesis, storage, release, metabolism reabsorption, or receptor binding of said biogenic amine or neurotransmitter.

5. The method of claim 4, wherein the compound of formula I binds to a receptor of a biogenic amine.

6. The method of claim 5, wherein the compound of formula I binds to a serotonin 5-HT_{2A} or 5-HT_{2C} receptor.

7. The method of claim 6, wherein the compound of formula I binds to a serotonin 5-HT_{2A} or 5-HT_{2C} receptor with an IC₅₀ of less than 1 μM.

8. The method of claim 1, wherein the compound of formula I binds to a σ1 receptor with an IC₅₀ of less than 1 μM.

9. The method of claim 1, wherein the c compound of formula I bind to a σ1 receptor and to at least one serotonin receptor selected from 5-HT_{2A} and 5-HT_{2C}.

10. The method of claim 1, wherein the disease or disorder of the central nervous system is selected from the group consisting of anxiety, depression, bipolar disorders, sleeping disorders, sexual disorders, psychosis, borderline psychosis, schizophrenia, migraine, personality disorders, obsessive-compulsive disorders, social phobia, of panic attacks, organic mental disorders in children, aggression, memory disorders, a personality disorders in elderly people, addiction, obesity, bulimia and other eating disorders, snoring, and premenstrual troubles.

11. The method of claim 1, wherein the damage to the central nervous system is caused by trauma, brain stroke, neurodegenerative diseases, cardiovascular disorders thrombosis, infarct or gastrointestinal disorders.

12. The method of claim 1 wherein X is O, S, or NR^{a}, wherein R^{a} is hydrogen or a substituent selected from the group consisting of C₁-C₃-alkyl, C₁-C₃-alkanoyl, C₇-C₁₀-aroyl, and C₇-C₁₀-arylalkyl.

13. The method of claim 1, wherein Y and Z are each independently selected from the group consisting of hydrogen, fluorine, chlorine, bromine, C₁-C₄-alkyl, halo-C₁-C₄-alkyl, hydroxy, C₁-C₄-alkoxy, trifluoromethoxy, C₁-C₄-alkanoyl, amino, amino-C₁-C₄-alkyl, N-(C₁-C₄-alkyl)amino, N,N-di(C₁-C₄-alkyl)amino, thiol, C₁-C₄-alkylthio, cyano and nitro.

14. The method of claim 1, wherein R¹ is selected from the group consisting of CHO, CH═CHOCOCH₃, (CH₂)ₘOH wherein m is an integer from 1 to 3; and a substituent of the formula II: 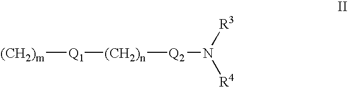
wherein
R³ and R⁴ are each independently hydrogen, C₁-C₄-alkyl, or aryl or
R³ and R⁴ taken together with the nitrogen atom to which they are attached form a heterocycle or heteroaryl group selected from the group consisting of morpholine-4-yl, piperidine-1-yl, pyrrolidine-1-yl, imidazole-1-yl and piperazine-1-yl;
m is an integer from 1 to 3;
n is an integer from 0 to 3; and
Q₁ and Q₂ are each independently oxygen or CH₂.

15. The method of claim 1, wherein the compound of formula I, is selected from the group consisting of:
1-methyl-1H-8-oxa-1,3-diaza-dibenzo[e,h]azulene-2-carbaldehyde;
1-methyl-1H-8-thia-1,3-diaza-dibenzo[e,h]azulene-2-carbaldehyde;
1-phenethyl-1H-8-oxa-1,3-diaza-dibenzo[e,h]azulene-2-carbaldehyde;
1-phenethyl-1H-8-thia-1,3-diaza-dibenzo[e,h]azulene-2-carbaldehyde;
1-(2-trimethylsilyl-ethoxymethyl)-1H-8-oxa-1,3-diaza-dibenzo[e,h]azulene-2-carbaldehyde;
1-(2-trimethylsilyl-ethoxymethyl)-1H-8-thia-1,3-diaza-dibenzo[e,h]azulene-2-carbaldehyde;
5-chloro-1-(2-trimethylsilyl-ethoxymethyl)-1H-8-oxa-1,3-diaza-dibenzo[e,h]azulene-2-carbaldehyde;
11-chloro-1-(2-trimethylsilyl-ethoxymethyl)-1H-8-oxa-1,3-diaza-dibenzo[e,h]azulene-2-carbaldehyde;
5-chloro-1-(2-trimethylsilyl-ethoxymethyl)-1H-8-thia-1,3-diaza-dibenzo[e,h]azulene-2-carbaldehyde;
11-chloro-1-(2-trimethylsilyl-ethoxymethyl)-1H-8-thia-1,3-diaza-dibenzo[e,h]azulene-2-carbaldehyde;
3-(1-phenethyl-1H-8-thia-1,3-diaza-dibenzo[e,h]azulen-2-yl)-acrylic acid methyl ester;
(1-methyl-1H-8-oxa-1,3-diaza-dibenzo[e,h]azulen-2-yl)-methanol;
(1-methyl-1H-8-thia-1,3-diaza-dibenzo[e,h]azulen-2-yl)-methanol;
(1-phenethyl-1H-8-oxa-1,3-diaza-dibenzo[e,h]azulen-2-yl)-methanol;
(1-phenethyl-1H-8-thia-1,3-diaza-dibenzo[e,h]azulen-2-yl)-methanol;
[1-(2-trimethylsilyl-ethoxymethyl)-1H-8-oxa-1,3-diaza-dibenzo[e,h]azulen-2-yl]-methanol;
[1-(2-trimethylsilyl-ethoxymethyl)-1H-8-thia-1,3-diaza-dibenzo[e,h]azulen-2-yl]-methanol;
[5-chloro-1-(2-trimethylsilyl-ethoxymethyl)-1H-8-oxa-1,3-diaza-dibenzo [e,h]azulen-2-yl]-methanol;
[11-chloro-1-(2-trimethylsilyl-ethoxymethyl)-1H-8-oxa-1,3-diaza-dibenzo[e,h]azulen-2-yl]-methanol;
[5-chloro-1-(2-trimethylsilyl-ethoxymethyl)-1H-8-thia-1,3-diaza-dibenzo[e,h]azulen-2-yl]-methanol;
[11-chloro-1-(2-trimethylsilyl-ethoxymethyl)-1H-8-thia-1,3-diaza-dibenzo[e,h]azulen-2-yl]-methanol;
3-(1-phenethyl-1H-8-thia-1,3-diaza-dibenzo[e,h]azulen-2-yl)-propane-1-ol;
dimethyl-[2-(1-methyl-1H-8-oxa-1,3-diaza-dibenzo[e,h]azulen-2-ylmethoxy)-ethyl]-amine;
dimethyl-[3-(1-methyl-1H-8-oxa-1,3-diaza-dibenzo [e,h]azulen-2-ylmethoxy)-propyl]-amine;
dimethyl-[2-(1-methyl-1H-8-thia-1,3-diaza-dibenzo[e,h] azulen-2-ylmethoxy)-ethyl]-amine;
dimethyl-[3-(1-methyl-1H-8-thia-1,3-diaza-dibenzo [e,h]azulen-2-ylmethoxy)-propyl]-amine;
dimethyl-[2-(1-phenethyl-1H-8-oxa-1,3-diaza-dibenzo[e,h]azulen-2-ylmethoxy)-ethyl]-amine;
dimethyl-[3-(1-phenethyl-1H-8-oxa-1,3-diaza-dibenzo[e,h]azulen-2-ylmethoxy)-propyl]-amine;
dimethyl-[2-(1-phenethyl-1H-8-thia-1,3-diaza-dibenzo [e,h] azulen-2-ylmethoxy)-ethyl]-amine;
dimethyl-[3-(1-phenethyl-1H-8-thia-1,3-diaza-dibenzo[e,h]azulen-2-ylmethoxy)-propyl]-amine;
dimethyl-{2-[1-(2-trimethylsilyl-ethoxymethyl)-1H-8-oxa-1,3-diaza-dibenzo[e,h]azulen-2-ylmethoxy]-ethyl}-amine;
dimethyl-[2-(1H-8-oxa-1,3-diaza-dibenzo[e,h]azulen-2-ylmethoxy)-ethyl]-amine;
dimethyl-{3-[1-(2-trimethylsilyl-ethoxymethyl)-1H-8-oxa-1,3-diaza-dibenzo[e,h]azulen-ylmethoxy]-propyl}-amine;
dimethyl-[3-(1H-8-oxa-1,3-diaza-dibenzo[e,h]azulen-2-ylmethoxy)-propyl]-amine;
3-[1-(2-trimethylsilyl-ethoxymethyl)-1H-8-oxa-1,3-diaza-dibenzo[e,h]azulen-2-ylmethoxy]-propylamine;
3-(1H-8-oxa-1,3-diaza-dibenzo[e,h]azulen-2-ylmethoxy)-propylamine;
dimethyl-{2-[1-(2-trimethylsilyl-ethoxymethyl)-1H-8-thia-1,3-diaza-dibenzo[e,h]azulen-2-ylmethoxy]-ethyl)}-amine;
dimethyl-[2-(1H-8-thia-1,3-diaza-dibenzo[e,h] azulen-2-ylmethoxy)-ethyl]-amine;
dimethyl-{3-[1-(2-trimethylsilyl-ethoxymethyl)-1H-8-thia-1,3-diaza-dibenzo[e,h]azulen-2-ylmethoxy]-propyl)}-amine;
dimethyl-[3-(1H-8-thia-1,3-diaza-dibenzo[e,h]azulen-2-ylmethoxy)-propyl]-amine;
{3-[5-chloro-1-(2-trimethylsilyl-ethoxymethyl)-1H-8-oxa-1,3-diaza-dibenzo[e,h]azulen-2-ylmethoxy]-propyl}-dimethyl-amine;
[3-(5-chloro-1H-8-oxa-1,3-diaza-dibenzo[e,h]azulen-2-ylmethoxy)-propyl]-dimethyl-amine;
3-[5-chloro-1-(2-trimethylsilyl-ethoxymethyl)-1H-8-oxa-1,3-diaza-dibenzo[e,h]azulen-2-ylmethoxy]-propylamine;
3-(5-chloro-1H-8-oxa-1,3-diaza-dibenzo[e,h]azulen-2-ylmethoxy)-propylamine;
{2-[11-chloro-1-(2-trimethylsilyl-ethoxymethyl)-1H-8-oxa-1,3-diaza-dibenzo[e,h]azulen-2-ylmethoxy]-ethyl)}-dimethyl-amine;
[2-(11-chloro-1H-8-oxa-1,3-diaza-dibenzo[e,h]azulen-2-ylmethoxy)-ethyl]-dimethyl-amine;
{3-[11-chloro-1-(2-trimethylsilyl-ethoxymethyl)-1H-8-oxa-1,3-diaza-dibenzo[e,h]azulen-2-ylmethoxy]-propyl}-dimethyl-amine;
[3-(11-chloro-1H-8-oxa-1,3-diaza-dibenzo[e,h]azulen-2-ylmethoxy)-propyl]-dimethyl-amine;
{2-[5-chloro-1-(2-trimethylsilyl-ethoxymethyl)-1H-8-thia-1,3-diaza-dibenzo[e,h]azulen-2-ylmethoxy]-ethyl)}-dimethyl-amine;
[2-(5-chloro-1H-8-thia-1,3-diaza-dibenzo[e,h]azulen-2-ylmethoxy)-ethyl]-dimethyl-amine;
{3 -[5-chloro-1-(2-trimethylsilyl-ethoxymethyl)-1H-8-thia-1,3-diaza-dibenzo[e,h]azulen-2-ylmethoxy]-propyl}-dimethyl-amine;
[3-(5-chloro-1H-8-thia-1,3-diaza-dibenzo[e,h]azulen-2-ylmethoxy)-propyl]-dimethyl-amine;
dimethyl-{3-[3-(1-phenethyl-1H-8-thia-1,3-diaza-dibenzo[e,h]azulen-2-yl)-propoxy]-propyl}-amine; and
a pharmaceutically acceptable salt or solvate thereof.
